(19) United States
(12) Patent Application Publication
(10) Pub. No.: US 2025/0387319 A1
(43) Pub. Date: Dec. 25, 2025

(54) Compositions and methods to reduce wrinkles, sagging skin, aging skin, spots, under eye puffiness and dark circles, and dry skin.

(71) Applicant: John Han Wu, Rowland Heights, CA (US)

(72) Inventor: John Han Wu, Rowland Heights, CA (US)

(21) Appl. No.: 19/231,521

(22) Filed: Jun. 8, 2025

Related U.S. Application Data
(60) Provisional application No. 63/663,195, filed on Jun. 24, 2024.

(51) Int. Cl. A61K 8/9789; A61K 8/44; A61Q 19/00; A61Q 19/02; A61Q 19/08

(52) U.S. Cl. CPC A61K 8/9789; A61K 8/44; A61Q 19/007; A61Q 19/02; A61Q 19/08

(57) ABSTRACT

A topical composition for improving skin conditions and appearance is disclosed. The composition includes a fenugreek extract and an amino acid, such as L-arginine, L-citrulline, L-arginine hydrochloride, L-citrulline malate, or combinations thereof. The composition may optionally include tartaric acid. Methods of using the composition for reducing wrinkles, sagging skin, aging skin, pigmented spots, under eye puffiness, under eye dark circles, and dry skin are also provided.

### CROSS-REFERENCE TO RELATED APPLICATIONS

This application claims the benefit of U.S. Provisional Application No. 63/663,195, filed Jun. 24, 2024, the entire disclosure of which is incorporated herein by reference.

### TECHNICAL FIELD

The present invention generally relates to compositions and methods for improving various skin conditions and the overall appearance of skin. More particularly, this invention pertains to novel topical compositions and their administration for enhancing skin health and aesthetics.

### BACKGROUND

Humans generally desire good skin conditions and a youthful appearance, free from visible signs of aging. Unfortunately, as individuals age, the skin undergoes various undesirable changes, increasingly exhibiting conditions such as wrinkles, pigmented spots (including age spots), sagging skin, overall signs of aging, under eye puffiness, under eye dark circles, and dry skin. These changes often diminish skin's aesthetic appeal and can impact self-perception.

While various cosmetic and dermatological treatments exist to address these concerns, many current solutions may suffer from limitations such as irritation, limited efficacy, safety concerns, or inconvenient administration methods. Therefore, there remains a significant need for non-irritating, highly effective, and safe compositions and methods for reliably improving a broad range of skin conditions and the overall appearance of skin.

### SUMMARY

The present disclosure addresses the need for effective topical compositions and methods to enhance skin health and aesthetics, particularly by targeting various common undesirable skin conditions. These conditions include, for example, wrinkles, sagging skin, aging skin, pigmented spots, under eye puffiness, under eye dark circles, and dry skin. The compositions and methods described herein offer significant improvements in these areas. In one aspect, the compositions comprise a fenugreek extract in combination with an amino acid component. The amino acid component includes at least one of L-arginine, L-citrulline, L-arginine hydrochloride, or L-citrulline malate. Methods for achieving the aforementioned skin improvements involve topically administering these compositions to an individual. In particular embodiments, the inventive compositions further include tartaric acid, which provides synergistic or additional benefits for improving the appearance of skin and addressing the specific conditions outlined herein. Corresponding methods of use are also provided.

### DETAILED DESCRIPTION

Skin aging involves loss of dermal collagen and elastin, increased matrix metalloproteinases (MMPs) and reactive oxygen species (ROS), and impaired microcirculation (Varani et al., 2006). Dry skin involves reduction in skin hydration and also decreased microcirculation.

The compositions of the present invention address these conditions through a dual-mechanism approach. The first component is a Trigonella foenum-graecum (fenugreek) extract. Fenugreek has shown anti-collagenase activity and increased collagen production in human fibroblast cells (Eaknai et al., 2022). Fenugreek can also significantly reduce the secretions of MMP1 and MMP9 compared to control cells after UV irradiation (Eaknai et al., 2022), thereby decreasing the photo aging effects of UV radiation on the skin. Rutin, one of fenugreek's main active components, has been shown to increase collagen as well (Eaknai et al., 2022). Trigonelline, another of fenugreek's main active component, can act as a phytoestrogen to stimulate estrogen receptors (Nguyen et al., 2024). An increase in estrogen in the skin leads to an increase in dermal hyaluronic acid and acid mucopolysaccharide levels, which in turn enhances skin hydration (Stevenson et al., 2007). Additionally, an increase in estrogen also boosts collagen production and thickens elastic fibers in the papillary dermis, thereby improving skin elasticity and reducing wrinkles (Stevenson et al., 2007).

The second essential component of the invention is an amino acid selected to enhance nitric oxide (NO) synthesis in the skin. This is achieved through the use of L-arginine, the direct substrate for nitric oxide synthase (eNOS), or L-citrulline, which serves as a highly efficient precursor to intracellular L-arginine (Arribas-López et al., 2021). L-arginine-driven NO production is critical for vasodilation, which improves cutaneous microcirculation and enhances the delivery of oxygen and nutrients to skin cells (Bode-Böger et al., 1998; Arribas-López et al., 2021). NO signaling is also understood to activate wound-healing pathways (Arribas-López et al., 2021). Arginine stimulates an increase in hydroxyproline, an amino acid essential for collagen synthesis, and arginine-based therapies have been shown to promote neovascularization and collagen deposition in tissue regeneration contexts (Arribas-López et al., 2021). These findings suggest that L-arginine can support the skin's structural integrity by fostering an environment conducive to matrix protein synthesis and tissue repair.

Critically, L-citrulline and L-arginine are intrinsically linked via the Arginine-Citrulline-NO Cycle. L-citrulline is converted to L-arginine by the enzymes argininosuccinate synthase and argininosuccinate lyase for subsequent utilization by endothelial cells within vessel walls to synthesize NO (Gonzalez et al., 2023). The conversion of L-citrulline to L-arginine for NO synthesis is well-documented in the art (Gonzalez et al., 2023). As such, both L-citrulline and L-arginine are suitable for practicing the present invention. Therefore, for the purposes of the present invention, providing L-citrulline is functionally an effective method of delivering L-arginine to the necessary intracellular location for NO production. A person skilled in the art, understanding this well-established biochemical pathway, would have a reasonable expectation that using either L-arginine or L-citrulline would result in the desired enhancement of NO synthesis and the consequent improvements in skin microcirculation and appearance. As such, L-arginine and L-citrulline are considered functionally equivalent embodiments for practicing the invention.

### Synergistic Dual-Mechanism For Anti-Aging Activity

The combination of Trigonella foenum-graecum (fenugreek) extract with an amino acid that serves as a substrate for nitric oxide synthesis, such as L-arginine or L-citrulline, presents a compelling mechanism for synergistic improvements in skin moisturization, elasticity, and wrinkle reduction. This potential synergy arises from the distinct yet complementary biochemical pathways modulated by each component.

Fenugreek and its components, like rutin and trigonelline, directly target the extracellular matrix by stimulating collagen and elastin synthesis and downregulating collagen-degrading enzymes like MMP-1 and MMP-9 in dermal fibroblasts (Eaknai et al., 2022). This action rebuilds and protects the skin's structural framework. Fenugreek's positive estrogenic effects also contributes to barrier hydration by increasing dermal hyaluronic acid and acid mucopolysaccharide levels (Eaknai et al., 2022).

In contrast, L-arginine and L-citrulline improve the underlying physiological environment necessary for skin health and repair. By serving as substrates for endothelial nitric oxide synthase (eNOS) (Gonzalez et al., 2023), they enhance NO bioavailability, which promotes microcirculation and improves the delivery of oxygen and nutrients. Besides microcirculation and angiogenesis, nitric oxide also stimulates the increased production of collagen, further enabling the collagen biosynthesis promoted by the fenugreek component (Sivaraj et al., 2023).

The technical rationale for synergy is based on these distinct mechanisms: fenugreek working to rebuild and protect the skin's extracellular matrix at a molecular and cellular level, while L-arginine/L-citrulline optimize the physiological conditions for these rebuilding processes to occur efficiently by focusing on improving the physiological environment through enhanced vascular perfusion and precursor availability as well as stimulating fibroblasts to increase collagen. The complementary actions suggest a strong potential for such outcomes.

From a conceptual standpoint for patentability, this dual-mechanism approach is non-obvious. Although fenugreek and L-arginine/L-citrulline are known individually for certain skin-beneficial and circulatory effects respectively, combining them to achieve potentially synergistic cosmetic outcomes by targeting distinct biochemical pathways (e.g., direct ECM modulation and MMP inhibition by fenugreek, versus NO-mediated microcirculation enhancement and nutrient support with collagen increase by arginine/citrulline) is not readily apparent and is not taught in the prior art.

The inclusion of tartaric acid, an alpha hydroxy acid (AHA), introduces a powerful third dimension to this synergistic approach. Tartaric acid primarily acts as a mild exfoliant, promoting the desquamation of dead skin cells from the stratum corneum (Almeman, 2024). This accelerated cell turnover reveals fresher, healthier skin beneath, leading to improved skin texture, reduced appearance of fine lines and wrinkles, and a more even skin tone (Almeman, 2024). Furthermore, AHAs like tartaric acid can stimulate collagen synthesis, thereby contributing to improved skin elasticity and firmness (Almeman, 2024). It also contributes to maintaining optimal skin pH (Almeman, 2024), which is crucial for barrier function to prevent dryness and to promote overall skin health.

By stimulating new collagen production, maintaining optimal skin pH for moisture retention, complementing fenugreek's barrier-hydrating estrogenic effects through increased dermal hyaluronic acid, and facilitating exfoliation for enhanced bioavailability of fenugreek and L-arginine/L-citrulline, tartaric acid significantly boosts the synergistic benefits of fenugreek extract and L-arginine/L-citrulline in the skin.

From a conceptual standpoint for patentability, this triple-mechanism approach is non-obvious. Although fenugreek, L-arginine/L-citrulline, and tartaric acid are known individually for certain skin-beneficial, circulatory, and exfoliating effects respectively, combining them to achieve potentially synergistic cosmetic outcomes by targeting distinct yet complementary biochemical pathways is not readily apparent and is not taught in the prior art.

The present inventor made the surprising discovery that usage of compositions with active ingredients containing a combination of fenugreek extract along with at least one of the amino acids L-arginine or L-citrulline or a combination thereof significantly reduced the appearance of wrinkles, pigmented spots, sagging skin, aging skin, under eye puffiness, under eye dark circles, and dry skin. The present inventor also surprisingly discovered that compositions containing a combination of fenugreek extract with tartaric acid along with at least one of the amino acids L-arginine or L-citrulline or a combination thereof, can effect a reduction in the appearance of wrinkles, pigmented spots, sagging skin, aging skin, under eye puffiness, under eye dark circles, and dry skin even slightly better than just the combination of fenugreek extract with at least one of the amino acids L-arginine or L-citrulline or a combination thereof.

The compositions and methods of the present invention meets the need of improving skin conditions and appearance without irritation.

As used herein, the term ‘fenugreek extract’ refers to an extract derived from Trigonella foenum-graecum, and may include, but is not limited to, aqueous extracts, alcoholic extracts (e.g., ethanol extracts), and glycerin extracts, prepared by methods as described herein.

The fenugreek extract may be derived from any part of the fenugreek plant, including but not limited to, the seeds, leaves, or stems. The extract may be prepared by various extraction methods known in the art, such as aqueous extraction, alcoholic (e.g., ethanol) extraction, or glycerin extraction. The fenugreek extract may be present in the final topical composition in an amount ranging from about 0.1% to about 25% by weight of the total composition.

In some embodiments, the fenugreek extract is an aqueous extract. An aqueous fenugreek extract may be prepared by macerating or refluxing fenugreek material (e.g., ground seeds) in water. For instance, fenugreek material may be steeped in hot water (e.g., from about 60° C. to about 100° C.) at a fenugreek to water ratio of from about 1:5 to about 1:30 (w/v or w/w) for a period of from about 1 hour to about 24 hours. The mixture is then typically filtered or centrifuged to separate the solid residue, and the liquid extract may optionally be concentrated (e.g., by evaporation) or spray-dried to yield a powdered extract. An aqueous extract may contain fenugreek solids ranging from about 10 wt % to about 80 wt % of the extract (especially if concentrated or powdered), with the remainder being water.

In other embodiments, the fenugreek extract is an alcoholic extract, such as an ethanol extract. An ethanol fenugreek extract may be prepared by macerating or refluxing fenugreek material (e.g., ground seeds) in an aqueous ethanol solution. The ethanol concentration in the extraction solvent may range from about 30% to about 96% (v/v) ethanol in water, with preferred concentrations from about 50% to about 80% (v/v) ethanol, and most preferably about 70% (v/v) ethanol. The fenugreek to solvent ratio during extraction may range from about 1:2 to about 1:20 (w/v or w/w), with preferred ratios from about 1:5 to about 1:10 (w/v or w/w). The extraction process typically involves contacting the fenugreek material with the ethanol solution for a period of from about 4 hours to about 72 hours, optionally with agitation or heating (e.g., from ambient temperature up to reflux temperature of the solvent). Following extraction, the mixture is typically filtered to remove solids, and the resulting liquid extract is usually concentrated (e.g., via evaporation under reduced pressure) to yield a final extract where the fenugreek solids (dry weight) comprise from about 5% to about 50% by weight of the final extract, with a more typical range being from about 10% to about 30% by weight. An exemplary ethanol extract may be prepared using 70% ethanol at a 1:10 w/v ratio of fenugreek to solvent, extracted at 60° C. for 6 hours, resulting in an extract containing approximately 12 wt % fenugreek solids after concentration.

In still other embodiments, the fenugreek extract is a glycerin extract. A glycerin fenugreek extract may be prepared by macerating fenugreek material (e.g., ground seeds) in a glycerin-containing solvent, which may optionally include water. The ratio of fenugreek to the glycerin-containing solvent may range from about 1:2 to about 1:10 (w/v or w/w). The extraction typically involves contacting the fenugreek material with the glycerin solvent for a period of from about 24 hours to about 7 days, optionally with gentle heating (e.g., from about 40° C. to about 60° C.) and agitation. After extraction, the mixture is filtered or centrifuged to separate the solid residue, yielding a liquid glycerin extract. Such an extract may contain fenugreek solids (dry weight) ranging from about 10 wt % to about 40 wt % of the extract, with the remainder being primarily glycerin and any co-solvent (e.g., water). In a specific embodiment, the fenugreek extract contains fenugreek at about 25 wt % of the weight of the fenugreek extract and contains glycerin at about 75 wt % of the fenugreek extract, which may be prepared by macerating 1 part by weight of fenugreek seeds in 3 parts by weight of a glycerin:water mixture (e.g., 80:20 glycerin:water) at 50° C. for 48 hours, followed by filtration.

The specific type of fenugreek extract and its concentration within the extract, as well as its final concentration in the topical composition, can be selected based on desired efficacy, formulation stability, and sensory characteristics.

As used herein, the amino acid component of the present compositions is selected from L-arginine, L-citrulline, L-arginine hydrochloride, L-citrulline malate, or combinations thereof. The amino acid is present in the final topical composition in an amount ranging from about 0.1% to about 25% by weight of the total composition, or more preferably from about 0.1% to about 10% by weight.

In some embodiments, the compositions further comprise tartaric acid. The tartaric acid may be present in an amount of from about 0.5% to about 2% by weight of the total composition. This component may provide enhanced benefits for improving skin conditions and appearance when combined with the fenugreek extract and amino acid components.

The components of the present invention may be combined in various concentrations. For example, a topical composition may comprise a fenugreek extract in an amount of from about 0.1% to about 25% by weight of the total composition, and an amino acid (as described above) in an amount of from about 0.1% to about 25% by weight of the total composition. In certain embodiments, this combination may additionally include tartaric acid in an amount of from about 0.5% to about 2% by weight of the total composition.

The compositions of the present invention can be formulated into various topical forms suitable for cosmetic or dermatological application. Non-limiting examples of such topical forms include a cream, lotion, serum, gel, ointment, foam, paste, shampoo, conditioner, soap, or mousse. The compositions may include pharmaceutically or cosmetically acceptable excipients, carriers, or diluents, such as water, humectants, emollients, emulsifiers, preservatives, and fragrances, as is well known in the art.

The present invention also provides methods for improving skin conditions and overall skin appearance. These methods involve topically applying a composition of the present invention to the skin of a human subject. The application should be for a period of time and in an amount sufficient to effect changes in the skin.

The application frequency may vary, but in some embodiments, the composition is applied twice daily for a period of about 28 days or longer to achieve desired results. The amount applied will depend on the area being treated and the concentration of active ingredients, as would be apparent to a person skilled in the art.

The improvement in skin appearance encompasses various specific conditions, including but not limited to, the reduction of wrinkles, alleviation of sagging skin, revitalization of aging skin, fading of pigmented spots, reduction of under eye puffiness, diminution of under eye dark circles, and amelioration of dry skin.

The following provides detailed embodiments and examples of the invention. Specific terms are used for clarity, but a person skilled in the relevant art will understand that other similar components can be used, and other compositions, or methods can be developed without straying from the spirit and scope of the invention.

Certain specific terms are utilized to explain particular embodiments of the invention's use, but these terms are not intended to restrict the broader applicability of the invention. Furthermore, singular terms like “a,” “an,” or “the” are intended to include their plural counterparts unless the context explicitly indicates otherwise.

It should also be noted that terms, as typically defined in standard dictionaries, are to be understood in a way that aligns with their contextual meaning within this application and the pertinent field, and should not be interpreted in an exaggerated or overly precise manner unless specifically stated. The language used in this invention's description serves solely to outline particular embodiments and is not meant to restrict the invention's scope.

The term “about,” as used in reference to a measurable value, such as an amount or concentration, is intended to encompass variations of up to ±10% of the specified value.

Described herein are compositions comprising skin-condition-improving mixtures of ingredients, and methods of improving skin conditions comprising administering an amount of a composition.

According to some embodiments of the present invention, provided herein are topical compositions.

In some embodiments, a topical composition of the present invention may comprise:
a Trigonella foenum-graecum (fenugreek) extract; and
an amino acid selected from L-arginine, L-citrulline, L-arginine hydrochloride, L-citrulline malate, or combinations thereof.

In some embodiments, a composition of the present invention may comprise fenugreek extract, wherein said fenugreek extract is present in an amount of from about 0.1% to about 25% by weight of the total composition.

In some embodiments, a composition of the present invention may comprise fenugreek extract, wherein said fenugreek extract is present in an amount of from about 0.1% to about 5% by weight of the total composition.

In some embodiments, a composition of the present invention may comprise an amino acid selected from L-arginine, L-citrulline, L-arginine hydrochloride, L-citrulline malate, or combinations thereof, in an amount of from about 0.1% to about 25% by weight of the total composition.

In some embodiments, a composition of the present invention may comprise an amino acid selected from L-arginine, L-citrulline, L-arginine hydrochloride, L-citrulline malate, or combinations thereof, in an amount of from about 0.1% to about 10% by weight of the total composition.

In some embodiments, a composition of the present invention may comprise fenugreek extract, wherein said fenugreek extract is present in an amount of from about 0.1% to about 25% by weight of the total composition, and an amino acid selected from L-arginine, L-citrulline, L-arginine hydrochloride, L-citrulline malate, or combinations thereof, wherein said amino acid is in an amount of from about 0.1% to about about 25% by weight of the total composition.

In some embodiments, a composition of the present invention may comprise fenugreek extract, wherein said fenugreek extract is present in an amount of from about 0.1% to about 25% by weight of the total composition, and an amino acid selected from L-arginine, L-citrulline, L-arginine hydrochloride, L-citrulline malate, or combinations thereof, wherein said amino acid is in an amount of from about 0.1% to about 25% by weight of the total composition, and a tartaric acid, wherein said tartaric acid is in an amount of from about 0.5% to about 2% by weight of the total composition.

In some embodiments, the compositions are in a topical form selected from a group consisting of: cream, lotion, serum, gel, ointment, foam, paste, shampoo, conditioner, soap, or mousse.

In some embodiments, the compositions can be used to improve skin conditions selected from the group consisting of: wrinkles, pigmented spots, sagging skin, aging skin, under eye puffiness, under eye dark circles, and dry skin.

In some embodiments, the compositions is applied twice daily for about 28 days or longer.

In some embodiments, provided herein is a method for improving skin conditions, comprising topically applying to the skin, for a period of time and in an amount sufficient to effect changes in the skin, of a composition comprising:
a Trigonella foenum-graecum (fenugreek) extract; and
an amino acid selected from L-arginine, L-citrulline, L-arginine hydrochloride, L-citrulline malate, or combinations thereof.

In some embodiments, provided herein is a method for improving skin conditions, comprising topically applying to the skin, for a period of time and in an amount sufficient to effect changes in the skin, of a composition comprising:
a Trigonella foenum-graecum (fenugreek) extract, wherein said fenugreek extract is in an amount of from about 0.1% to about 25% by weight of the total composition; and
an amino acid selected from L-arginine, L-citrulline, L-arginine hydrochloride, L-citrulline malate, or combinations thereof.

In some embodiments, provided herein is a method for improving skin conditions, comprising topically applying to the skin, for a period of time and in an amount sufficient to effect changes in the skin, of a composition comprising:
a Trigonella foenum-graecum (fenugreek) extract, wherein said fenugreek extract is in an amount of from about 0.1% to about 5% by weight of the total composition; and
an amino acid selected from L-arginine, L-citrulline, L-arginine hydrochloride, L-citrulline malate, or combinations thereof.

In some embodiments, provided herein is a method for improving skin conditions, comprising topically applying to the skin, for a period of time and in an amount sufficient to effect changes in the skin, of a composition comprising:
a Trigonella foenum-graecum (fenugreek) extract; and
an amino acid selected from L-arginine, L-citrulline, L-arginine hydrochloride, L-citrulline malate, or combinations thereof, wherein said amino acid is in an amount of from about 0.1% to about 25% by weight of the total composition.

In some embodiments, provided herein is a method for improving skin conditions, comprising topically applying to the skin, for a period of time and in an amount sufficient to effect changes in the skin, of a composition comprising:
a Trigonella foenum-graecum (fenugreek) extract; and
an amino acid selected from L-arginine, L-citrulline, L-arginine hydrochloride, L-citrulline malate, or combinations thereof, wherein said amino acid is in an amount of from about 0.1% to about 10% by weight of the total composition.

In some embodiments, provided herein is a method for improving skin conditions, comprising topically applying to the skin, for a period of time and in an amount sufficient to effect changes in the skin, of a composition comprising:
a Trigonella foenum-graecum (fenugreek) extract, wherein said fenugreek extract is in an amount of from about 0.1% to about 25% by weight of the total composition; and
an amino acid selected from L-arginine, L-citrulline, L-arginine hydrochloride, L-citrulline malate, or combinations thereof, wherein said amino acid is in an amount of from about 0.1% to about 25% by weight of the total composition.

In some embodiments, provided herein is a method for improving skin conditions, comprising topically applying to the skin, for a period of time and in an amount sufficient to effect changes in the skin, of a composition comprising:
a Trigonella foenum-graecum (fenugreek) extract; and
an amino acid selected from L-arginine, L-citrulline, L-arginine hydrochloride, L-citrulline malate, or combinations thereof; and a tartaric acid, wherein said tartaric acid is in an amount of from about 0.5% to about 2% by weight of the total composition.

In some embodiments, provided herein is a method for improving skin conditions, comprising topically applying to the skin, for a period of time and in an amount sufficient to effect changes in the skin, of a composition comprising:
a Trigonella foenum-graecum (fenugreek) extract, wherein said fenugreek extract is in an amount of from about 0.1% to about 25% by weight of the total composition; and
an amino acid selected from L-arginine, L-citrulline, L-arginine hydrochloride, L-citrulline malate, or combinations thereof, wherein said amino acid is in an amount of from about 0.1% to about 25% by weight of the total composition; and a tartaric acid, wherein said tartaric acid is in an amount of from about 0.5% to about 2% by weight of the total composition.

In some embodiments, provided herein is a method comprising topically applying to the skin, for a period of time and in an amount sufficient to effect changes in the skin, of a composition comprising a fenugreek extract and an amino acid comprising at least one of the group consisting of L-arginine, L-citrulline, L-arginine hydrochloride, or L-citrulline malate, in order to improve skin conditions selected from the group consisting of wrinkles, pigmented spots, sagging skin, aging skin, under eye puffiness, under eye dark circles, and dry skin. In other embodiments, a tartaric acid is added to said fenugreek extract and said amino acid.

In some embodiments, provided herein is a method comprising a composition, wherein said composition is in a topical form selected from a group consisting of: cream, lotion, serum, gel, ointment, foam, paste, shampoo, conditioner, soap, or mousse.

In some embodiments, provided herein is a method comprising topically applying to the skin, for a period of time and in an amount sufficient to effect changes in the skin, a composition comprising a fenugreek extract, a tartaric acid, and an amino acid comprising at least one of the group consisting of L-arginine, L-citrulline, L-arginine hydrochloride, or L-citrulline malate, wherein said composition is in a topical form selected from a group consisting of: cream, lotion, serum, gel, ointment, foam, paste, shampoo, conditioner, soap, or mousse.

In some embodiments, provided herein is a method comprising a composition, wherein said composition is applied twice daily for about 28 days or longer.

In some embodiments, provided herein is a method comprising topically applying to the skin, for a period of time and in an amount sufficient to effect changes in the skin, a composition comprising a fenugreek extract, and an amino acid comprising at least one of the group consisting of L-arginine, L-citrulline, L-arginine hydrochloride, or L-citrulline malate, wherein said composition is applied twice daily for about 28 days or longer. Optionally, in other embodiments, a tartaric acid is added to said fenugreek extract and said amino acid, and the total composition is applied twice daily for about 28 days or longer.

The compositions of the present invention can be formulated in various cosmetic products using various delivery systems and carrier bases. In some embodiments, the composition is delivered topically and is formulated as a water-in-oil emulsion and as an oil-in-water emulsion. In some embodiments, the compositions are delivered topically and are formulated through delivery systems and carrier bases such as a cream, serum, solution, lotion, gel, ointment, wax, aerosol spray, powder, paste, foam, balm, shampoo, conditioner, after shave, soap, and mousse. In some embodiments, the compositions for dietary consumption is formulated as a powder.

The selection of the cosmetically acceptable carrier in the present composition depends on the specific formulation type. For instance, ointments, pastes, creams, or gels may utilize carbohydrates, starches, thickeners, tragacanth, fatty materials, paraffins, waxes, animal or vegetable fats, cellulose derivatives, humectants, polyethylene glycols, emollients, silicones, minerals, bentonite, silica, talc, or metal oxides.

Cosmetically acceptable carriers for lotions and emulsions may include solvents and emulsifiers such as water, butylene glycol, ethanol, preservatives, and vegetable oils.

Excipient options for this formulation include sugars (lactose), anti-caking agents (talc, silica), and absorption enhancers (aluminum hydroxide, calcium silicate, polyamide powder)

Compositions in form of emulsions are prepared by first mixing the water soluble and oil soluble ingredients separately, with emulsifiers added to the oil soluble portion and thickening agents added to the water soluble portion. For water-in-oil emulsions the oil soluble portion is then added slowly to the water soluble portion while the water soluble portion is being mixed using a homogenizer machine. For oil-in-water emulsions the water soluble portion is added slowly to the oil soluble portion while the oil soluble portion is being mixed using a homogenizer machine.

Compositions in form of solutions are created by adding ingredients separately to water and stirring until the ingredients are completely dissolved, then mixing all ingredients together.

In another aspect of the present invention, the composition further comprises one or more excipient selected from the group consisting of water, hydrocarbon oils, mineral oil, silicone oil, silicone fluid, silicone compounds, petrolatum, humectants, surfactants, emulsifiers, wetting agents, rheology modifiers, emollients, saccharides, fatty alcohols, waxes, nitrogen compounds, conditioning polymers, and rheology modifiers.

In the compositions of the present invention, additional ingredients can be added, including, but not limited to, examples such as pharmaceutical active ingredients, anti aging rejuvenators, micronutrients, antioxidants, skin softeners, UV protectors, sunscreen ingredients, whitening agents, pigmentation reducing agents, endocrine regulators, pain relievers, vitamins, minerals, herbal extracts, numbing agents, anti acne agents, bacterial inhibitors, fungal suppressors, plant-derived compounds, medicinal ingredients, additives, preservatives, thickeners, skin softeners, hydrating agents, skin barrier enhancers, moisture retainers, synthetic polymers, calming components, pigments, cellulite reducing agents, wrinkle reducing agents, and fragrances.

### EXAMPLES

The compositions described herein can be readily formulated using well-known techniques or commercially available components, for example from Sigma Aldrich company (based in St. Louis, Missouri, USA). One skilled in the art of composition formulation would recognize that numerous variations can be implemented without departing from the inventive concept. While the present application does not exhaustively describe all possible ingredient sources or formulation techniques, the general knowledge of a skilled formulator provides sufficient guidance to enable preparation of the claimed compositions.

The following examples are just illustrations to help explain the invention, and do not limit the claims provided in this invention.

While accuracy in all numbers (amounts, weights, percentages, etc.) is aimed for, there's a possibility of minor errors or variations.

When the phrase “effective amount” is mentioned, it refers to a quantity of a composition from the present invention that produces a beneficial response in an individual. Experts in the field will understand that the beneficial effects do not have to be comprehensive or curative, as long as they offer some advantage to the individual.

The different parts and features described in these examples and embodiments can be used together in various combinations.

Example compositions of the present invention include, but are not limited to, those provided in the following tables.

Fenugreek extract is available as a liquid from many vendors on Amazon, or from, for example, Pipingrock.com and hawaiipharm.com, as a super concentrated liquid with fenugreek itself at a 25% wt of a 1:3 (fenugreek:solvent) fenugreek glycerin extract. Instead of glycerin, ethanol can also be used as the solvent without detracting from fenugreek's effects, and many vendors on Amazon and other online sources also sell ethanolic fenugreek extract, but fenugreek glycerin extract is preferred. Xanthan gum is available from many vendors from Walmart or Amazon, or from lotioncrafter.com or makingcosmetics.com. L-arginine, L-citrulline, L-arginine hydrochloride, L-citrulline malate, and tartaric acid are all available from many vendors on Amazon, or from vitaminshoppe.com, bulksupplements.com, iherb.com, swansonvitamins.com, or sigmaaldrich.com. Distilled water is available from many vendors on Amazon, or from Walmart or Target.

### Example 1

The following describes preparation of a 100 gram solution of L-citrulline and liquid fenugreek extract (wherein the fenugreek itself is about 25 wt % of a fenugreek glycerin extract).

TABLE 1

| Ingredient | % w/w | grams | |
|---|---|---|---|
| Liquid | 0.1% (~0.025% | 0.1 gram | |
| Fenugreek | fenugreek, ~0.075% | (~0.025 | |
| extract | glycerin) | gram fenugreek, ~0.075 | |
| | | gram glycerin) | |
| L-citrulline (powder) | 1% | 1 | gram |
| Water (distilled) | 98.9% | 98.9 | gram |

### Procedure:

Add L-citrulline powder to distilled water, stir the mixture until it completely dissolves. Add liquid fenugreek extract to the mixture, stir until it completely dissolves.

The percentages of the liquid fenugreek extract and L-citrulline can be adjusted higher or lower as desired, with the percentage of water adjusted higher or lower at the end in order to make 100% w/w. L-arginine can be used instead of L-citrulline, with the procedure remaining the same.

### Example 2

The following describes preparation of a 100 gram solution of L-citrulline and liquid fenugreek extract (wherein the fenugreek itself is about 25 wt % of a fenugreek glycerin extract).

TABLE 2

| Ingredient | % w/w | grams |
|---|---|---|
| Liquid | 5% (~1.25% | 5 gram |
| Fenugreek | fenugreek, ~3.75% | (~1.25 gram |
| extract | glycerin) | fenugreek, ~3.75 |
| | | gram glycerin) |
| L-citrulline (powder) | 10% | 10 gram |
| Water (distilled) | 85% | 85 gram |

### Procedure:

Add L-citrulline powder to distilled water, stir the mixture until it completely dissolves. Add liquid fenugreek extract to the mixture, stir until it completely dissolves.

The percentages of the liquid fenugreek extract and L-citrulline can be adjusted higher or lower as desired, with the percentage of water adjusted higher or lower at the end in order to make 100% w/w. L-arginine can be used instead of L-citrulline, with the procedure remaining the same.

### Example 3

The following describes preparation of a 100 gram solution of L-citrulline and liquid fenugreek extract (wherein the fenugreek itself is about 25 wt % of a fenugreek glycerin extract).

TABLE 3

| Ingredient | % w/w | grams |
|---|---|---|
| Liquid | 25% (~6.25% | 25 gram |
| Fenugreek | fenugreek, ~18.75% | (~6.25 gram |
| extract | glycerin) | fenugreek, ~18.75 |
| | | gram glycerin) |
| L-citrulline (powder) | 25% | 25 gram |
| Water (distilled) | 50% | 50 gram |

### Procedure:

Add L-citrulline powder to distilled water, stir the mixture until it completely dissolves. Add liquid fenugreek extract to the mixture, stir until it completely dissolves.

The percentages of the liquid fenugreek extract and L-citrulline can be adjusted higher or lower as desired, with the percentage of water adjusted higher or lower at the end in order to make 100% w/w. L-arginine can be used instead of L-citrulline, with the procedure remaining the same.

### Example 4

The following describes preparation of a 100 gram solution of L-citrulline, tartaric acid, and liquid fenugreek extract (wherein the fenugreek itself is about 25 wt % of a fenugreek glycerin extract).

TABLE 4

| Ingredient | % w/w | grams |
|---|---|---|
| Liquid | 5% (~1.25% | 5 gram |
| Fenugreek | fenugreek, ~3.75% | (~1.25 gram |
| extract | glycerin) | fenugreek, ~3.75 |
| | | gram glycerin) |
| L-citrulline (powder) | 10% | 10 gram |
| Tartaric acid (powder) | 1% | 1 gram |
| Water (distilled) | 84% | 84 gram |

### Procedure:

Add L-citrulline powder to distilled water, stir the mixture until it completely dissolves. Add tartaric acid to the mixture, stir the mixture until it completely dissolves. Add liquid fenugreek extract to the mixture, stir until it completely dissolves.

The percentages of the liquid fenugreek extract, tartaric acid, and L-citrulline can be adjusted higher or lower as desired, with the percentage of water adjusted higher or lower at the end in order to make 100% w/w. L-arginine can be used instead of L-citrulline, with the procedure remaining the same.

### Example 5

The following describes preparation of a 100 gram gel of L-citrulline and liquid fenugreek extract (wherein the fenugreek itself is about 25 wt % of a fenugreek glycerin extract).

TABLE 5

| Ingredient | % w/w | grams | |
|---|---|---|---|
| Liquid | 0.1% (~0.025% | 0.1 gram | |
| Fenugreek | fenugreek, ~0.075% | (~0.025 gram | |
| extract | glycerin) | fenugreek, ~0.075 | |
| | | gram glycerin) | |
| L-citrulline (powder) | 1% | 1 | gram |
| Xanthan gum (powder) | 1% | 1 | gram |
| Phenoxyethanol (powder) | 0.5% | 0.5 | gram |
| Water (distilled) | 97.4% | 97.4 | gram |

### Procedure:

Divide the water into 2 equal parts. Add L-citrulline powder to part 1 of the water, stir the part 1 mixture until it completely dissolves. Add liquid fenugreek extract to the part 1 mixture, stir until it completely dissolves. Add phenoxyethanol to the part 1 mixture, stir until it completely dissolves. Take the xanthan gum, and while using a homogenizer machine or a shearing mixing machine to mix the part 2 of the water, add the xanthan gum very gradually by sprinkling it onto part 2 of the water while the water is being mixed. After all of the xanthan gum has been added to part 2 of the water, continue mixing the resulting part 2 mixture until all of the xanthan gum has been dissolved into the water without any clumps. Then, add part 1 mixture and part 2 mixture together gradually using a homogenizer machine, and mix until the resulting mixture is homogenous.

The percentages of the liquid fenugreek extract and L-citrulline can be adjusted higher or lower as desired. L-arginine can be used instead of L-citrulline, with the procedure remaining the same. Xanthan gum is used as a thickener to make the mixture into a gel, and can be replaced with any other thickener, with its percentage adjusted as needed to make a thicker or thinner mixture. Phenoxyethanol is used as a preservative, and can be replaced with any other preservative. The percentage of water is adjusted higher or lower at the end in order to make 100% w/w. One skilled in the art can convert the foregoing gel into a cream by substituting the aqueous vehicle with a standard oil-in-water emulsifier system (e.g., cetyl alcohol, stearic acid, polysorbate 60) and adjusting the water phase accordingly, without needing further inventive effort. Likewise, a lotion can be prepared by reducing the thickener concentration and increasing the water content to achieve a pourable viscosity.

### Example 6

The following describes preparation of a 100 gram gel of L-citrulline and liquid fenugreek extract (wherein the fenugreek itself is about 25 wt % of a fenugreek glycerin extract).

TABLE 6

| Ingredient | % w/w | grams | |
|---|---|---|---|
| Liquid | 5% (~1.25% | 5 gram | |
| Fenugreek | fenugreek, ~3.75% | (~1.25 gram | |
| extract | glycerin) | fenugreek, ~3.75 | |
| | | gram glycerin) | |
| L-citrulline (powder) | 10% | 10 | gram |
| Xanthan gum (powder) | 1% | 1 | gram |
| Phenoxyethanol (liquid) | 0.5% | 0.5 | gram |
| Water (distilled) | 83.5% | 83.5 | gram |

### Procedure:

Divide the water into 2 equal parts. Add L-citrulline powder to part 1 of the water, stir the part 1 mixture until it completely dissolves. Add liquid fenugreek extract to the part 1 mixture, stir until it completely dissolves. Add phenoxyethanol to the part 1 mixture, stir until it completely dissolves. Take the xanthan gum, and while using a homogenizer machine or a shearing mixing machine to mix the part 2 of the water, add the xanthan gum very gradually by sprinkling it onto part 2 of the water while the water is being mixed. After all of the xanthan gum has been added to part 2 of the water, continue mixing the resulting part 2 mixture until all of the xanthan gum has been dissolved into the water without any clumps. Then, add part 1 mixture and part 2 mixture together gradually using a homogenizer machine, and mix until the resulting mixture is homogenous.

The percentages of the liquid fenugreek extract and L-citrulline can be adjusted higher or lower as desired. L-arginine can be used instead of L-citrulline, with the procedure remaining the same. Xanthan gum is used as a thickener to make the mixture into a gel, and can be replaced with any other thickener, with its percentage adjusted as needed to make a thicker or thinner mixture. Phenoxyethanol is used as a preservative, and can be replaced with any other preservative. The percentage of water is adjusted higher or lower at the end in order to make 100% w/w. One skilled in the art can convert the foregoing gel into a cream by substituting the aqueous vehicle with a standard oil-in-water emulsifier system (e.g., cetyl alcohol, stearic acid, polysorbate 60) and adjusting the water phase accordingly, without needing further inventive effort. Likewise, a lotion can be prepared by reducing the thickener concentration and increasing the water content to achieve a pourable viscosity.

### Example 7

The following describes preparation of a 100 gram gel of L-citrulline and liquid fenugreek extract (wherein the fenugreek itself is about 25 wt % of a fenugreek glycerin extract).

TABLE 7

| Ingredient | % w/w | grams | |
|---|---|---|---|
| Liquid | 25% (~6.25% | 25 gram | |
| Fenugreek | fenugreek, ~18.75% | (~6.25 gram | |
| extract | glycerin) | fenugreek, ~18.75 | |
| | | gram glycerin) | |
| L-citrulline (powder) | 25% | 25 | gram |
| Xanthan gum (powder) | 1% | 1 | gram |
| Phenoxyethanol (liquid) | 0.5% | 0.5 | gram |
| Water (distilled) | 48.5% | 48.5 | gram |

### Procedure:

Divide the water into 2 equal parts. Add L-citrulline powder to part 1 of the water, stir the part 1 mixture until it completely dissolves. Add liquid fenugreek extract to the part 1 mixture, stir until it completely dissolves. Add phenoxyethanol to the part 1 mixture, stir until it completely dissolves. Take the xanthan gum, and while using a homogenizer machine or a shearing mixing machine to mix the part 2 of the water, add the xanthan gum very gradually by sprinkling it onto part 2 of the water while the water is being mixed. After all of the xanthan gum has been added to part 2 of the water, continue mixing the resulting part 2 mixture until all of the xanthan gum has been dissolved into the water without any clumps. Then, add part 1 mixture and part 2 mixture together gradually using a homogenizer machine, and mix until the resulting mixture is homogenous.

The percentages of the liquid fenugreek extract and L-citrulline can be adjusted higher or lower as desired. L-arginine can be used instead of L-citrulline, with the procedure remaining the same. Xanthan gum is used as a thickener to make the mixture into a gel, and can be replaced with any other thickener, with its percentage adjusted as needed to make a thicker or thinner mixture. Phenoxyethanol is used as a preservative, and can be replaced with any other preservative. The percentage of water is adjusted higher or lower at the end in order to make 100% w/w. One skilled in the art can convert the foregoing gel into a cream by substituting the aqueous vehicle with a standard oil-in-water emulsifier system (e.g., cetyl alcohol, stearic acid, polysorbate 60) and adjusting the water phase accordingly, without needing further inventive effort. Likewise, a lotion can be prepared by reducing the thickener concentration and increasing the water content to achieve a pourable viscosity.

### Example 8

The following describes preparation of a 100 gram gel of L-citrulline and liquid fenugreek extract (wherein the fenugreek itself is about 25 wt % of a fenugreek glycerin extract).

TABLE 8

| Ingredient | % w/w | grams | |
|---|---|---|---|
| Liquid | 5% (~1.25% | 5 gram | |
| Fenugreek | fenugreek, ~3.75% | (~1.25 gram | |
| extract | glycerin) | fenugreek, ~3.75 | |
| | | gram glycerin) | |
| L-citrulline (powder) | 10% | 10 | gram |
| Tartaric acid (powder) | 1% | 1 | gram |
| Xanthan gum (powder) | 1% | 1 | gram |
| Phenoxyethanol (liquid) | 0.5% | 0.5 | gram |
| Water (distilled) | 82.5% | 82.5 | gram |

### Procedure:

Divide the water into 2 equal parts. Add L-citrulline powder to part 1 of the water, stir the part 1 mixture until it completely dissolves. Add tartaric acid powder to part 1 mixture, stir the part 1 mixture until it completely dissolves. Add liquid fenugreek extract to the part 1 mixture, stir until it completely dissolves. Add phenoxyethanol to the part 1 mixture, stir until it completely dissolves. Take the xanthan gum, and while using a homogenizer machine or a shearing mixing machine to mix the part 2 of the water, add the xanthan gum very gradually by sprinkling it onto part 2 of the water while the water is being mixed. After all of the xanthan gum has been added to part 2 of the water, continue mixing the resulting part 2 mixture until all of the xanthan gum has been dissolved into the water without any clumps. Then, add part 1 mixture and part 2 mixture together gradually using a homogenizer machine, and mix until the resulting mixture is homogenous.

The percentages of the liquid fenugreek extract and L-citrulline can be adjusted higher or lower as desired. L-arginine can be used instead of L-citrulline, with the procedure remaining the same. Tartaric acid percentage can be adjusted higher or lower as desired. Xanthan gum is used as a thickener to make the mixture into a gel, and can be replaced with any other thickener, with its percentage adjusted higher or lower as needed to make a thicker or thinner mixture. Phenoxyethanol is used as a preservative, and can be replaced with any other preservative. The percentage of water is adjusted higher or lower at the end in order to make 100% w/w. One skilled in the art can convert the foregoing gel into a cream by substituting the aqueous vehicle with a standard oil-in-water emulsifier system (e.g., cetyl alcohol, stearic acid, polysorbate 60) and adjusting the water phase accordingly, without needing further inventive effort. Likewise, a lotion can be prepared by reducing the thickener concentration and increasing the water content to achieve a pourable viscosity.

### Example 9

### Objective:

To evaluate, in a within-subject clinical design, the efficacy of four topical gel formulations containing Trigonella foenum-graecum (fenugreek) extract and L-citrulline, with and without tartaric acid, on multiple parameters of skin appearance, including wrinkle depth, pigmented spots, skin sagging, overall aging appearance, under-eye puffiness, under-eye dark circles, and skin dryness (TEWL). An additional fifth gel formulation, a Glycerin-Only Control Gel (1 wt % xanthan gum, 19 wt % glycerin, 0.5 wt % phenoxyethanol as preservative, water 79.5 wt %), acts as control.

### Study Population:

Twenty-seven (27) healthy adult volunteers (ages 30-60, all genders) exhibiting visible signs of facial aging (wrinkles, fine lines, pigmented spots, sagging skin), neck aging (wrinkles, fine lines, pigmented spots, sagging skin), under-eye concerns (puffiness, dark circles), and dry skin were enrolled. Subjects were not using other topical actives on their face during the 28-day study.

### Formulations:

Each formulation was prepared as a simple gel using a standard xanthan gum

based gel base (e.g., 1 wt % xanthan gum, plus water, and 0.5 wt % phenoxyethanol as preservative). All percentages are by weight of total composition:
Low-Dose Gel:
   0.10 wt % fenugreek extract (˜0.025% fenugreek, ˜0.075% glycerin)
   1.0 wt % L-citrulline
   1 wt % xanthan gum (gel base)
   0.5 wt % phenoxyethanol
   q.s. to 100 wt % with purified water
Mid-Dose Gel:
   5.0 wt % fenugreek extract (˜1.25% fenugreek, ˜3.75% glycerin)
   10.0 wt % L-citrulline
   1 wt % xanthan gum (gel base)
   0.5 wt % phenoxyethanol
   q.s. to 100 wt % with purified water
High-Dose Gel:
   25.0 wt % fenugreek extract (˜6.25% fenugreek, ˜18.75% glycerin)
   25.0 wt % L-citrulline
   1 wt % xanthan gum (gel base)
   0.5 wt % phenoxyethanol
   q.s. to 100 wt % with purified water
Mid+Tartaric Gel:
   5.0 wt % fenugreek extract (˜1.25% fenugreek, ˜3.75% glycerin)
   10.0 wt % L-citrulline
   1.0 wt % tartaric acid
   1 wt % xanthan gum (gel base)
   0.5 wt % phenoxyethanol
   q.s. to 100 wt % with purified water
Control Gel:
   19.0 wt % glycerin
   1 wt % xanthan gum (gel base)
   0.5 wt % phenoxyethanol
   q.s. to 100 wt % with purified water

Each gel was mixed under gentle agitation until homogenous and stored at room temperature, per the procedures described in example 1 through example 8.

### Control (Baseline and Glycerin-Only Control):

Subjects refrained from applying any product to all mapped study areas for 24 hours prior to baseline measurements (Day 0). The Day 0 values for each endpoint served as each subject's own baseline control. Thereafter, a glycerin-only control gel (1 wt % xanthan gum, 19 wt % glycerin, balance purified water) was applied twice daily to Area 7 (right neck). Baseline (Day 0) measurements in Area 7 and Day 28 measurements in Area 7 confirm the effect of glycerin alone. All other areas (Areas 1-6) were compared to their respective Day 0 baselines to evaluate the fenugreek/L-citrulline containing gels and the fenugreek/L-citrulline/tartaric-acid containing gels.

### Anatomical Treatment Areas & Dosing:

Each subject's face was partitioned into four treatment zones (each ˜4 cm²) and two under-eye zones (each ˜2 cm²), and one control zone (˜4 cm²):
Area 1 (Right Forehead including glabellar region): Low-Dose Gel
Area 2 (Left Forehead including glabellar region): Mid-Dose Gel
Area 3 (Right Cheek): High-Dose Gel
Area 4 (Left Cheek): Mid+Tartaric Gel
Area 5 (Right Under Eye): Low-Dose Gel
Area 6 (Left Under Eye): Mid-Dose Gel
Area 7 (Right Neck): Glycerin Only Control Gel

Subjects applied approximately 0.1 g of gel to each forehead/cheek/neck area twice daily (morning, evening) and 0.05 g to each under-eye area twice daily, for 28 days.

### Endpoints & Measurement Methods:

The following describes how each skin condition is measured.
1. Wrinkle-Depth Reduction (Areas 1-4, plus area 7):
   Method: 3D fringe-projection imaging (PRIMOS) at Day 0 and Day 28.
   Calculation: percent change between day 28 and baseline day 0
2. Pigmented-Spot Reduction (Areas 1-4, plus area 7):
   Method: Cross-polarized digital photography+automated image-analysis software.
   Calculation: Percent decrease in total pigmented-spot area versus baseline day 0
3. Sagging-Skin Reduction (Areas 1-4, plus area 7):
   Method: 3D topographic analysis of fold depth
   Calculation: Percent decrease in fold depth relative to baseline day 0
4. Overall Aging Appearance Improvement (Areas 1-4, plus area 7):
   Method: Blinded dermatologist grading on a 5-point scale (1=no improvement; 5=very marked improvement) at Day 0 and Day 28.
   Conversion: Scale difference converted to percentage improvement from baseline. Percent improvement=((average graded score−1)/4)×100
5. Under-Eye Puffiness Reduction (Area 5):
   Method: Optical coherence tomography (OCT) to measure periorbital tissue thickness at Day 0 vs. Day 28.
   Calculation: Percent decrease relative to baseline.
6. Under-Eye Dark-Circle Lightening (Area 6):
   Method: Colorimetric analysis (L* value) under standardized lighting; higher L* indicates lighter skin.
   Calculation: Percent improvement from baseline, change in L *.
7. Dryness Improvement (TEWL Decrease) (Areas 1-4, plus area 7):
   Method: Open-chamber evaporimetry at each area at Day 0 and Day 28.
   Calculation: Percent decrease in TEWL from baseline.

### Statistical Analysis:

For each endpoint and each area, mean percent change was calculated over all 27 subjects. A paired t-test compared Day 0 vs. Day 28 values for each area, with statistical significance defined as p<0.05 for all endpoints. Each percentage change is rounded to the nearest whole number.

### Results:

The following shows the respective results in Mean % Change, with n =27.
1. Wrinkle-Depth Reduction:
   Area 1 (Low-Dose): 11%
   Area 2 (Mid-Dose): 14%
   Area 3 (High-Dose): 18%
   Area 4 (Mid+Tartaric): 16%
   Area 7 (Glycerin control on right neck): 3%
2. Pigmented-Spot Reduction:
   Area 1 (Low-Dose): 10%
   Area 2 (Mid-Dose): 15%
   Area 3 (High-Dose): 20%
   Area 4 (Mid+Tartaric): 17%
   Area 7 (Glycerin control on right neck): 0%
3. Sagging-Skin Reduction:
   Area 1 (Low-Dose): 11%
   Area 2 (Mid-Dose): 15%
   Area 3 (High-Dose): 19%
   Area 4 (Mid+Tartaric): 17%
   Area 7 (Glycerin control on right neck): 0%
4. Overall Aging Appearance Improvement:
   Area 1 (Low-Dose): 10%
   Area 2 (Mid-Dose): 14%
   Area 3 (High-Dose): 18%
   Area 4 (Mid+Tartaric): 16%
   Area 7 (Glycerin control on right neck): 1%
5. Under-Eye Puffiness Reduction (Area 5-Low-Dose):
   Area 5: 9%
6. Under-Eye Dark-Circle Lightening (Area 6-Mid-Dose):
   Area 6: 14%
7. Dryness Improvement (TEWL Decrease):
   Area 1 (Low-Dose): 12%
   Area 2 (Mid-Dose): 16%
   Area 3 (High-Dose): 20%
   Area 4 (Mid+Tartaric): 18%
   Area 7 (Glycerin control on right neck): 4%

### Safety:

No adverse reactions or irritation were reported for any of the formulations over the 28-day application period.

### Conclusion:

These results demonstrate a clear dose-response for all facial endpoints (Areas 1-4): increasing fenugreek and citrulline concentrations yield progressively greater improvements in wrinkle depth, pigmented spots, skin sagging, overall aging appearance, and TEWL. The inclusion of 1 wt % tartaric acid in the Mid+Tartaric Gel (Area 4) produces a synergistic enhancement beyond the Mid-Dose Gel (Area 2). Under-eye data confirm that Low-Dose Gel (Area 5) reduces puffiness by 9%, and Mid-Dose Gel (Area 6) lightens dark circles by 14%. In contrast, the glycerin-only gel (Area 7) applied to the right neck produced minimal improvements, underscoring the contribution of the fenugreek/L-citrulline and the fenugreek/L-citrulline/tartaric-acid combinations to the stated skin improvement benefits.

I claim:

1. A topical composition for improving the appearance of skin, comprising:
a Trigonella foenum-graecum (fenugreek) extract; and
an amino acid selected from L-arginine, L-citrulline, L-arginine hydrochloride, L-citrulline malate, or combinations thereof.

2. The composition according to claim 1, wherein said fenugreek extract is present in an amount of from about 0.1% to about 25% by weight of the total composition.

3. The composition according to claim 1, wherein said amino acid is present in an amount of from about 0.1% to about 25% by weight of the total composition.

4. The composition according to claim 1, wherein said fenugreek extract is present in an amount of from about 0.1% to about 5% by weight of the total composition.

5. The composition according to claim 1, wherein said amino acid is present in an amount of from about 0.1% to about 10% by weight of the total composition.

6. The composition according to claim 1, wherein said fenugreek extract is present in an amount of from about 0.1% to about 25% by weight of the total composition, and said amino acid is present in an amount of from about 0.1% to about 25% by weight of the total composition.

7. The composition according to claim 6, further comprising tartaric acid, in an amount of from about 0.5% to about 2% by weight of the total composition.

8. The composition according to claim 1, wherein said composition is in a topical form selected from cream, lotion, serum, gel, ointment, foam, paste, shampoo, conditioner, soap, or mousse.

9. The composition according to claim 1, wherein said composition is applied twice daily for about 28 days or longer.

10. The composition according to claim 1, wherein said improvement in skin appearance comprises at least one of reduction in appearance of: wrinkles, pigmented spots, sagging skin, aging skin, under eye puffiness, under eye dark circles, and dry skin.

11. A method of improving skin conditions and skin appearance in a human subject, comprising topically applying to the skin, for a period of time and in an amount sufficient to effect changes in the skin, of a composition comprising:
a Trigonella foenum-graecum (fenugreek) extract; and
an amino acid selected from L-arginine, L-citrulline, L-arginine hydrochloride, L-citrulline malate, or combinations thereof.

12. The method according to claim 11, wherein said composition contains said fenugreek extract in an amount of from about 0.1% to about 25% by weight of the total composition.

13. The method according to claim 11, wherein said composition contains said amino acid in an amount of from about 0.1% to about 25% by weight of the total composition.

14. The method according to claim 11, wherein said fenugreek extract is present in an amount of from about 0.1% to about 5% by weight of the total composition.

15. The method according to claim 11, wherein said amino acid is present in an amount of from about 0.1% to about 10% by weight of the total composition.

16. The method according to claim 11, wherein said composition contains said fenugreek extract in an amount of from about 0.1% to about 25% by weight of the total composition, and said composition also contains said amino acid in an amount of from about 0.1% to about 25% by weight of the total composition.

17. The method according to claim 16, wherein said composition further contains tartaric acid, in an amount of from about 0.5% to about 2% by weight of the total composition.

18. The method according to claim 11, wherein said composition is in a topical form selected from cream, lotion, serum, gel, ointment, foam, paste, shampoo, conditioner, soap, or mousse.

19. The method according to claim 11, wherein said composition is applied twice daily to the skin for about 28 days or longer.

20. The method according to claim 11, wherein said improvement in skin appearance comprises at least one of reduction in appearance of: wrinkles, pigmented spots, sagging skin, aging skin, under eye puffiness, under eye dark circles, and dry skin.
